(19) United States
(12) Patent Application Publication
(10) Pub. No.: US 2024/0317668 A1
(43) Pub. Date: Sep. 26, 2024

(54) A PROCESS FOR PRODUCING ALKYL TRIFLUOROACETATES

(71) Applicant: BASF SE, Ludwigshafen (DE)

(72) Inventors: Joachim Gebhardt, Ludwigshafen (DE); Manfred Ehresmann, Ludwigshafen (DE); Marcus Zeumke, Ludwigshafen (DE); Roland Goetz, Ludwigshafen (DE); Martin Sesing, Ludwigshafen (DE); Daniel Maximilian Knoll, Ludwigshafen (DE)

(21) Appl. No.: 18/578,134

(22) PCT Filed: Jul. 4, 2022
(86) PCT No.: PCT/EP2022/068370

§ 371 Date: Jan. 10, 2024

(30) Foreign Application Priority Data
Jul. 14, 2021 (EP) 21185587.9

(51) Int. Cl. C07C 67/08; C07C 67/54; C07D 271/06

(52) U.S. Cl. CPC C07C 67/08; C07C 67/54; C07D 271/06

(57) ABSTRACT

A process for producing alkyl trifluoroacetates The present invention relates to a process for producing alkyl trifluoroacetates by reacting trifluoroacetic acid with an alkyl alcohol R—OH in an aqueous solution, and wherein the alkyl trifluoroacetate is separated from the reaction medium by means of direct rectification. In one aspect the present invention relates to a process for recovering trifluoroacetic acid in the form of alkyl trifluoroacetates from aqueous waste-water streams of preceding chemical transformations.

The present invention relates to a process for producing alkyl trifluoroacetates (ATFA) by reacting trifluoroacetic acid (TFA) with an alkyl alcohol R—OH in an aqueous solution, and wherein the alkyl trifluoroacetate is separated from the reaction medium by means of direct rectification. In one aspect the present invention relates to a process for recovering trifluoroacetic acid in the form of alkyl trifluoroacetates from aqueous waste-water streams of preceding chemical transformations leading to substituted 3-aryl-5-trifluoromethyl-1,2,4-oxadiazoles. These compounds are known to be useful for controlling phytopathogenic fungi, for example from WO 2015/185485 A1 and WO 2017/211649 A1.

Prior art references typically suggest producing ATFA by reacting TFA with an alcohol in the absence of water and employing a strong acid in amounts that may be catalytic, stoichiometric, or in excess. Suitable acids are, for example, concentrated sulfuric acid or acidic resins. If the acid is used in stoichiometric amounts or in excess, it may also serve to retain water formed during the condensation reaction between TFA and the alcohol.

The absence of water in those reactions is crucial because ATFA is highly reactive towards nucleophiles and thus prone to hydrolysis, in particular with esters in which the alkyl residue is small (C₁-C₃-alcohols). Oftentimes, ATFA is (re-)used in chemical transformations such as acetylation reactions, which require water-free conditions.

Moreover, TFA forms a maximum homogeneous azeotropic mixture with water, which has a higher boiling point than TFA alone. A separation of TFA from water by rectification is therefore impossible.

V. D. Talnikar et al. (Chemical Engineering Communications 2017, 204:356-364) report on a process designed for the recovery of TFA from industrial waste-water streams by reactive distillation. The authors emphasize the problem that TFA forms azeotropic mixtures with water. To solve this problem they propose a process, which involves a technically demanding set-up as it conceptually requires the continuous addition of an aqueous TFA solution to the top of a distillation column. Apart from its complexity, other disadvantages of the described process are that recovery rates reportedly reach only up to 80%, which is not satisfactory in industrial settings. Further, the methyl trifluoroacetate (MTFA) obtained in the process is not free of water and still contains TFA.

JP 11343267 discloses a process comprising reacting TFA dissolved in an aqueous medium containing an alkyl alcohol to obtain ATFA. The authors in this reference likewise refer to the problem that TFA forms azeotropic mixtures with water, suggesting that this problem may be solved by using water-insoluble solvents (dichloromethane, n-octanol) in a two-phased reaction mixture. In those cases, where n-octanol was used, the alcohol served as the reactant and as water-insoluble solvent providing for the physical separation of the n-octanol ester from the aqueous phase containing the TFA. The reaction involves intermediary steps such as manually separating the (n-octanol) ester from the aqueous medium and then recovering the ester, for example by a sequence of transesterification with a desired lower molecular weight alcohol, e.g. ethanol, and subsequent separation and purification of the ethyl ester by distillation.

From an economic point of view it is desirable in industrial large-scale operations for the production of ATFA or the recovery of TFA from aqueous mixtures to provide simple and robust processes with high space-time yield, that do not require the use of other organic solvents than the reactants, and that do not involve multiple steps.

In view of this, it was an object of the present invention to overcome the disadvantages of the procedures presented in the prior art and to provide a more efficient process, which enables the preparation of ATFA on an industrial scale, in a conventional apparatus, high yielding and without contamination of the product obtained by rectification, herein referred to as “the distillate”, with TFA or intolerable amounts of water.

The inventors found that essentially quantitative conversion of TFA can be achieved according to the process of the present invention, wherein ATFA is rectified directly from an aqueous reaction medium, optionally together with an excess of alkyl alcohol. Surprisingly, the distillate contained only negligible amounts of TFA and water, which means that the ATFA is stable upon storage, i.e. it does not suffer hydrolysis, and the ATFA is sufficiently dry to be used in chemical transformations that ask for very low levels of water.

Accordingly, the present invention relates to a process for producing alkyl trifluoroacetates of formula I,

CF₃—C(═O)—O—R I

wherein the variable R is C₁-C₆-alkyl or C₃-C₆-cycloalkyl; the process comprising reacting trifluoroacetic acid with an alkyl alcohol R—OH in an aqueous solution, wherein variable R in alkyl alcohol R—OH has the same meaning as R in the alkyl trifluoroacetate of formula I; and wherein the alkyl trifluoroacetate I is separated from the reaction medium by means of direct rectification.

The term “direct rectification” as used herein shall mean that a suitable rectification column or apparatus is connected to the reaction vessel containing the aqueous reaction mixture and that the rectification process commences from the reaction vessel either without timely delay after charging the reaction vessel with the aqueous mixture of the reactants or after allowing time for the reaction to proceed.

In one embodiment of the present invention alkyl alcohol R—OH is methanol, ethanol, n-propanol, iso-propanol, n-butanol, iso-butanol, sec-butanol, tert-butanol, cyclopentanol, or cyclohexanol; or mixtures thereof.

In one embodiment alkyl alcohol R—OH is methanol, n-propanol, iso-propanol, n-butanol, iso-butanol, sec-butanol, tert-butanol, cyclopentanol, or cyclohexanol; or mixtures thereof.

In one embodiment alkyl alcohol R—OH is methanol, ethanol, n-propanol, or iso-propanol; or mixtures thereof.

In one embodiment alkyl alcohol R—OH is methanol, n-propanol, or iso-propanol; or mixtures thereof.

In a preferred embodiment R—OH is methanol or ethanol; or mixtures thereof.

In another preferred embodiment R—OH is a mixture of methanol and ethanol.

In a particularly preferred embodiment R—OH is methanol.

In one aspect of the process of the present invention the amount of alkyl alcohol R—OH in the reaction mixture is equal to or more than the amount of TFA. In a preferred embodiment the amount of alkyl alcohol is 2 equivalents or more than the amount of TFA in the solution.

The process of the present invention is typically carried out at atmospheric pressure.

In one aspect the process of the present invention is carried out so that the product is rectified off of the aqueous mixture, which is heated to a temperature between 20° C. and 100° C., at atmospheric pressure.

In another aspect the process of the present invention is carried out so that the product is rectified off of the aqueous mixture, which is heated to a temperature between 40° C. and 100° C., at atmospheric pressure.

In one embodiment the process of the present invention is carried out at a pH of the aqueous medium, which is equal to or lower than 7.

In another embodiment the process of the present invention is carried out at a pH of the aqueous medium, which is equal to or lower than 0.

In a preferred aspect the process of the present invention is carried out at a pH of the aqueous medium, which is equal to or lower than 7, and wherein alkyl alcohol R—OH is methanol, ethanol, n-propanol, iso-propanol, n-butanol, iso-butanol, sec-butanol, tert-butanol, cyclopentanol, or cyclohexanol; or mixtures thereof.

In a preferred aspect the process of the present invention is carried out at a pH of the aqueous medium, which is equal to or lower than 7, and wherein the rectification is carried out at a temperature of the reaction mixture between 20° C. and 100° C., at atmospheric pressure, and wherein alkyl alcohol R—OH is methanol, ethanol, n-propanol, iso-propanol, n-butanol, iso-butanol, sec-butanol, tert-butanol, cyclopentanol, or cyclohexanol; or mixtures thereof.

In a preferred aspect the process of the present invention is carried out at a pH of the aqueous medium, which is equal to or lower than 0, and wherein alkyl alcohol R—OH is methanol, ethanol, n-propanol, or iso-propanol; or mixtures thereof.

In a preferred aspect the process of the present invention is carried out at a pH of the aqueous medium, which is equal to or lower than 0, and wherein the rectification is carried out at a temperature of the reaction mixture between 20° C. and 100° C., at atmospheric pressure, and wherein alkyl alcohol R—OH is methanol, ethanol, n-propanol, or iso-propanol; or mixtures thereof.

In another preferred aspect the process of the present invention is carried out at a pH of the aqueous medium, which is equal to or lower than 0, and wherein alkyl alcohol R—OH is methanol.

In another preferred aspect the process of the present invention is carried out at a pH of the aqueous medium, which is equal to or lower than 0, wherein the rectification is carried out at a temperature of the reaction mixture between 40° C. and 100° C., at atmospheric pressure, and wherein alkyl alcohol R—OH is methanol.

In order to allow for low levels of water in the distillate suitable means for rectification may be considered in the process according to the present invention. A person skilled in the art will appreciate that the rectification apparatus must provide a certain number of theoretical separator stages, or plates, that will suit the problem at hand. Clearly, this number depends on the physical properties of the reactants as well as the desired water content of the distillate. Therefore, to be able to control the concentration of water in the distillate, the minimum count of theoretical separation steps is chosen so as to optimize the number of plates (or column height) and the energy consumption.

In one embodiment the present invention relates to a process for recovering trifluoroacetic acid in the form of alkyl trifluoroacetates from aqueous waste-water streams of preceding chemical transformations leading to substituted 3-aryl-5-trifluoromethyl-1,2,4-oxadiazoles that are known to be useful for controlling phytopathogenic fungi, for example from WO 2015/185485 A1 and WO 2017/211649 A1.

Accordingly, in one aspect the present invention relates to a process for preparing oxadiazole compounds of formula II,

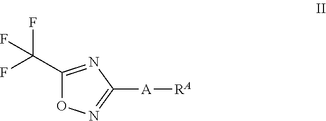

wherein
A is phenyl or a 5- or 6-membered aromatic heterocycle; wherein the ring member atoms of the aromatic heterocycle include besides carbon atoms 1, 2, 3, or 4 heteroatoms selected from N, O, and S as ring member atoms with the provision that the heterocycle cannot contain 2 contiguous atoms selected from O and S; and wherein A is further unsubstituted or further substituted with additional n identical or different radicals R^{a}; wherein n is 0, 1, 2, 3, or 4;
   R^{a} is independently selected from the group consisting of halogen, cyano, C₁-C₆-alkyl, C₁-C₆-haloalkyl, C₁-C₆-alkoxy, and C₁-C₆-haloalkoxy;
R^{A} is methyl, chloromethyl, hydroxymethyl, trichloromethyl, ethyl, iso-propyl, OH, SH, cyano, halogen, CH₂F, CHF₂, 2,2,2-trifluoroethyl, cyclopropyl, —C(═O)H, —C(═NOR₂)H, —C(═O)OH, —C(═O)OR¹, —C(═W)N(R¹R²), —CR³R⁴—N(R¹R²), —CR³R⁴—OR¹, —C(═NR¹)R³, —C(═O)R³, —CR³R⁴—C(═O)OH, —CR³R⁴—C(═O)R¹, —CR³R⁴—C(═W)N(R¹R²), —O—CR³R⁴—C(═O)OH, —O—CR³R⁴—C(═O)R¹, —O—CR³R⁴C(═W)N(R¹R²), —CR³R⁴—N(R²)—C(═W)R¹, —CR³R⁴—S(═O)₂R¹, or —CR³R⁴—N(R²)—S(═O)₂R¹; wherein
W is O or S;
R² is hydrogen, C₁-C₆-alkyl, C₂-C₆-alkenyl, C₂-C₆-alkynyl, C₁-C₆-alkoxy, C₃-C₁₁-cycloalkyl, —C(═O)—C₁-C₆-alkyl, —C(═O)—C₃-C₁₁-cycloalkyl, or —C(═O)—O—C₁-C₆-alkyl; and wherein any of the aliphatic or cyclic groups in R² are unsubstituted or substituted with 1, 2, 3, or up to the maximum possible number of identical or different radicals selected from the group consisting of halogen, hydroxy, oxo, cyano, C₁-C₆-alkyl, C₁-C₆-alkoxy, and C₃-C₁₁-cycloalkyl;
R¹ is C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, C₁-C₆-alkylamino, diC₁-C₆-alkylamino, —C(═O)—C₁-C₆-alkyl, —C(═O)—O—C₁-C₆-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl, or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms with the provision that the heterocycle cannot contain 2 contiguous atoms selected from O and S; and wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O, and S as ring member atoms with the provision that the heterocycle cannot contain 2 contiguous atoms selected from O and S; and wherein any of the above-mentioned aliphatic or cyclic groups are unsubstituted or substituted with 1, 2, 3, or up to the maximum possible number of identical or different groups R^{1a}; or
R¹ and R², together with the nitrogen atom to which they are attached, form a saturated or partially unsaturated mono- or bicyclic 3- to 10-membered heterocycle, wherein the heterocycle includes beside one nitrogen atom and one or more carbon atoms no further heteroatoms or 1, 2 or 3 further heteroatoms independently selected from N, O, and S as ring member atoms with the provision that the heterocycle cannot contain 2 contiguous atoms selected from O and S; and wherein the heterocycle is unsubstituted or substituted with 1, 2, 3, 4, or up to the maximum possible number of identical or different groups R^{1a}; wherein
   R^{1a} is halogen, oxo, cyano, NO₂, OH, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, —NHSO₂-C₁-C₄-alkyl, —C(═O)—C₁-C₄-alkyl, —C(═O)—O—C₁-C₄-alkyl, C₁-C₆-alkylsulfonyl, hydroxyC₁-C₄-alkyl, —C(═O)—NH₂, —C(═O)—NH(C₁-C₄-alkyl), C₁-C₄-alkylthio-C₁-C₄-alkyl, aminoC₁-C₄-alkyl, C₁-C₄-alkylamino-C₁-C₄-alkyl, diC₁-C₄-alkylamino-C₁-C₄-alkyl, aminocarbonyl-C₁-C₄-alkyl, or C₁-C₄-alkoxy-C₁-C₄-alkyl;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy; or
R³ and R⁴ together with the carbon atom to which they are bound form a cyclopropyl group;
the process comprising the following steps:
step 1: reacting an amidoxime of formula III,

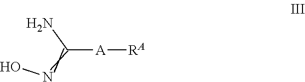

wherein the variables A and R^{A} are as defined above for compounds of formula II, with an alkyl trifluoroacetate of formula I,

CF₃—C(═O)—O—R I

wherein R is C₁-C₆-alkyl or C₃-C₆-cycloalkyl, in the presence of a base;

step 2: adding water to the reaction mixture obtained in step 1 and then separating amidoxime IlI from the aqueous phase;

step 3: treating the combined aqueous phases obtained in step 2 with an auxiliary acid, whereas the amount of the auxiliary acid is equivalent to or more than the amount of base used in step 1;

step 4: separating the alkyl trifluoroacetate I from the aqueous phases obtained in step 3 by rectification according to the process as defined or preferably defined herein.

Processes for the production of oxadiazole compounds II by reaction of amidoximes III with ATFA were disclosed in PCT/EP2021/052256 and in PCT/EP2021/052257.

A typical procedure involves, that the amidoxime III obtained in step 2, i.e. after addition of water, is separated from the aqueous phase by filtration, whereas the filter cake is further washed with water in order to collect all of the TFA. The combined aqueous phases are then further used in step 3 and 4.

To achieve complete conversion in step 1 of the above process the reaction requires the addition of at least 2 equivalents of ATFA to the amidoxime, based on the amount of amidoxime. Since ATFA is expensive and because high levels of TFA in aqueous waste-streams is a concern for disposal from an ecological perspective, the advantage of this process over processes of the prior art lies in the possibility to recover ATFA in dry form and without TFA in step 3 and feed it back into the process in step 1 without the need of further treatment.

The reaction in step 1 of the process for the production of oxadiazole compounds II generates alcohol R—OH as a by-product. This alcohol is carried through to step 4, which means that there is, in theory, no need to add further alcohol R—OH to the combined aqueous phases in step 4 to accomplish the recovery of ATFA.

In one aspect of the present invention process step 1 is conducted in the presence of an auxiliary solvent.

The term “auxiliary solvent” as used herein refers to a solvent, which is either identical with alcohol R—OH as defined herein or it may be selected from a dipolar organic solvent which is not identical with alcohol R—OH. Suitable dipolar organic solvents are, for example, ethers (diethylether, dibutylether, tert-butylmethylether, ethylene glycol dimethyl ether, ethylene glycol, diethyl ether, diethylene glycol dimethyl ether, 2-methyltetrahydrofuran, tetrahydrofuran, dioxane, diethylene, glycol monomethyl- or monoethyl ether), N-substituted lactams (N-methylpyrrolidone), carboxamides (N,N-dimethylformamide, N,N-dimethylacetamide), acyclic ureas (dimethyl imidazolinum), sulphoxides, sulphones (dimethyl sulfoxide, dimethyl sulfone, tetramethylene sulfoxide, tetramethylene sulfone).

In a preferred aspect of the present invention the auxiliary solvent in process step 1 for the production of oxadiazole compounds II comprises an alkyl alcohol, preferably an alkyl alcohol R—OH as defined or preferably defined herein.

In another preferred aspect of the present invention the auxiliary solvent in process step 1 for the production of oxadiazole compounds II comprises methanol or ethanol; particularly the auxiliary solvent is methanol or mixtures of methanol and ethanol.

The term “auxiliary acid” as used herein refers to an acid, which is not identical with TFA and has a pKa below 0.

In one aspect of the present invention the auxiliary acid in step 3 of the process for the production of oxadiazole compounds II is hydrochloric acid, sulfuric acid, phosphoric acid, or nitric acid.

In a preferred aspect of the present invention the auxiliary acid in step 3 of the process for the production of oxadiazole compounds II is hydrochloric acid or sulfuric acid.

In one embodiment of the process for the production of oxadiazole compounds II, the base in step 1 comprises sodium or potassium C₁-C₆-alkoxylates. In a further aspect of the process for the production of oxadiazole compounds II, the base in step 1 is sodium methoxide or sodium ethoxide.

In one aspect of the present invention variable A in compounds of formula II is phenyl. In one embodiment of the present invention radical R^{a} in compounds of formula II is halogen, C₁-C₆-alkyl, C₁-C₆-haloalkyl, C₁-C₆-alkoxy, or C₁-C₆-haloalkoxy; particularly fluorine.

In still another aspect n is 1 and R^{a} is fluorine in compounds of formula II.

In a preferred embodiment the variable n is 0 in compounds of formula II.

One aspect the present invention relates to a process for the production of oxadiazole compounds as defined above, wherein the amidoxime is of formula III.b,

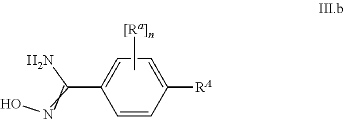

wherein n is 0 or 1, and the meaning of R^{a} and R^{A} is as defined herein for compounds of formula II, to obtain oxadiazole compounds of formula II.b, wherein the variables n, R^{a}, and R^{A} have the meaning as defined for compounds of formula III.b.

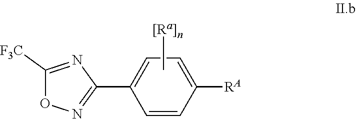

In another embodiment n is 1 and R^{a} is fluorine in compounds of II.b and III.b.

In a preferred embodiment n is 0 in compounds of formula II.b and III.b.

In one embodiment the variables in compounds of formula II, II.b, IlI and IlI.b have the following meaning:
R^{A} is fluorine;
n is 0 or 1;
R is methyl, chloromethyl, hydroxymethyl, trichloromethyl, —C(═O)H, —C(═NOR₂)H, —C(═O)OH, OH, SH, cyano, halogen, —C(═O)NR¹R², —CH₂—N(R²)—C(═O)R¹, —CH₂—N(R²)—S(═O)₂R¹,

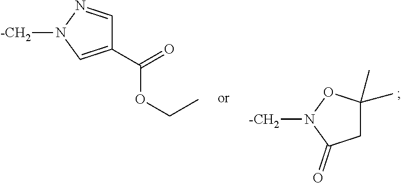

R¹ is methyl, ethyl, n-propyl, iso-propyl, n-butyl, sec-butyl, iso-butyl, cyclopropyl, 2-methoxyiminoethyl, bicyclo[1.1.1]pentan-1-yl, or phenyl; and wherein the phenyl group is unsubstituted or substituted with 1, 2, 3 or up to the maximum possible number of identical or different radicals selected from the group consisting of fluorine, chlorine, cyano, methyl, ethyl, methoxy, trifluoromethyl, trifluoromethoxy, difluoromethyl, difluoromethoxy, and cyclopropyl;
R² is hydrogen, methyl, ethyl, methoxy, ethoxy, or cyclopropyl.

In a further embodiment the variables in compounds of formula II, II.b, IlI and IlI.b have the following meaning:
R^{A} is fluorine;
n is 0 or 1;
R is methyl, —C(═O)OH, —C(═O)NR¹R², —CH₂—N(R²)—C(═O)R¹, —CH₂—N(R²)—S(═O)₂R¹,

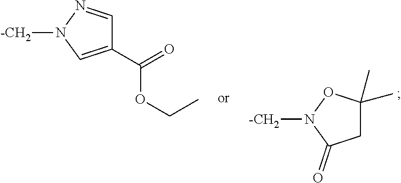

R¹ is C₁-C₆-alkly, phenyl, or cyclopropyl, wherein the phenyl ring is unsubstituted or substituted with 1, 2, 3, or 4 identical or different groups selected from halogen;
R² is hydrogen, methyl, ethyl, methoxy, ethoxy, or cyclopropyl.

In yet another embodiment the variables in compounds of formula II, II.b, IlI and III.b have the following meaning:
R^{A} is fluorine;
n is 0 or 1;
R is —CH₂—N(R²)—C(═O)R¹, —CH₂—N(R²)—S(═O)₂R¹,

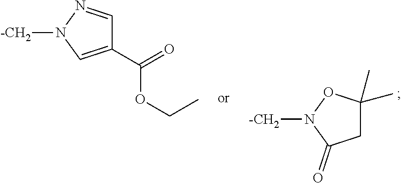

R¹ is C₁-C₆-alkly, or cyclopropyl;
R² is hydrogen, methyl, methoxy, ethoxy, or cyclopropyl.

In another embodiment the variables in compounds of formula II, II.b, IlI and III.b have the following meaning:
R^{A} is fluorine;
n is 0 or 1;
R is methyl, —C(═O)OH, or —C(═O)NR¹R²;
R¹ is methyl or phenyl, wherein the phenyl ring is unsubstituted or substituted with 1, 2, 3, or 4 identical or different groups selected from halogen;
R² is hydrogen, methyl, ethyl, methoxy, or ethoxy.

In still another embodiment the variables in compounds of formula II, II.b, IlI and III.b have the following meaning:
n is 0;
R^{A} is —C(═O)NR¹R²;
R¹ is methyl, 2-methoxyiminoethyl, bicyclo[1.1.1]pentan-1-yl, 2-fluoro-phenyl, 4-fluoro-phenyl, or 2,4-difluorophenyl; in particular methyl or 2-fluoro-phenyl;
R² is hydrogen.

In a further embodiment of the present invention the compound of formula II.b as defined or preferably defined herein, and wherein n is 0 and R^{A} is —C(═O)NR¹R², is used to obtain a compound of formula IV,

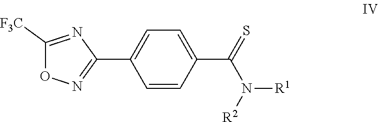

as described in WO 2019/020451 A1 and WO 2017/211649 A1 and the references cited therein; particularly for the preparation of compounds of formula IV, wherein R¹ is methyl or 2-fluoro-phenyl and R² is hydrogen.

The general expression “compound I” as used herein is equivalent to the expression “compounds of formula l”. Accordingly, for example, the expression “alkyl trifluoroacetate of formula I” as used herein is equivalent to the expression “alkyl trifluoroacetate I”.

In the definitions of the variables given above, collective terms are used which are generally representative for the substituents in question.

The term “Cn-Cm” indicates the number of carbon atoms possible in each case in the substituent or substituent moiety in question.

The term “halogen” refers to fluorine, chlorine, bromine and iodine.

The term “oxo” refers to an oxygen atom ═O, which is bound to a carbon atom or sulfur atom, thus forming, for example, a ketonyl —C(═O)— or sulfinyl —S(═O)— group.

The term “C₁-C₆-alkyl” refers to a straight-chained or branched saturated hydrocarbon group having 1 to 6 carbon atoms, for example methyl, ethyl, propyl, 1-methylethyl, butyl, 1-methylpropyl, 2-methylpropyl, and 1,1-dimethylethyl.

The term “C₂-C₆-alkenyl” refers to a straight-chain or branched unsaturated hydrocarbon radical having 2 to 6 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond, such as ethynyl, 1-propynyl, 2-propynyl (propargyl), 1-butynyl, 2-butynyl, 3-butynyl, 1-methyl-2-propynyl.

The term “C₁-C₆-haloalkyl” refers to a straight-chained or branched alkyl group having 1 to 6 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂-C₂F₅, CF₂-C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “C₁-C₆-alkoxy” refers to a straight-chain or branched alkyl group having 1 to 6 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy.

The term “C₁-C₆-haloalkoxy” refers to a C₁-C₆-alkoxy group as defined above, wherein some or all of the hydrogen atoms may be replaced by halogen atoms as mentioned above, for example, OCH₂F, OCHF₂, OCF₃, OCH₂Cl, OCHCI₂, OCCI₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy.

The terms “phenyl-C₁-C₄-alkyl or heteroaryl-C₁-C₄-alkyl” refer to alkyl having 1 to 4 carbon atoms (as defined above), wherein one hydrogen atom of the alkyl radical is replaced by a phenyl or hetereoaryl radical respectively.

The term “C₁-C₄-alkoxy-C₁-C₄-alkyl” refers to alkyl having 1 to 4 carbon atoms (as defined above), wherein one hydrogen atom of the alkyl radical is replaced by a C₁-C₄-alkoxy group (as defined above). Likewise, the term “C₁-C₄-alkylthio-C₁-C₄-alkyl” refers to alkyl having 1 to 4 carbon atoms (as defined above), wherein one hydrogen atom of the alkyl radical is replaced by a C₁-C₄-alkylthio group.

The term “C₁-C₆-alkylthio” as used herein refers to straight-chain or branched alkyl groups having 1 to 6 carbon atoms (as defined above) bonded via a sulfur atom. Accordingly, the term “C₁-C₆-haloalkylthio” as used herein refers to straight-chain or branched haloalkyl group having 1 to 6 carbon atoms (as defined above) bonded through a sulfur atom, at any position in the haloalkyl group.

The term “C₁-C₄-alkoxyimino” refers to a divalent imino radical (C₁-C₄-alkyl-O—N═) carrying one C₁-C₄-alkoxy group as substituent, e.g. methylimino, ethylimino, propylimino, 1-methylethyl-imino, butylimino, 1-methylpropylimino, 2-methylpropylimino, 1,1-dimethylethylimino and the like.

The term “C₁-C₆-alkoxyimino-C₁-C₄-alkyl” refers to alkyl having 1 to 4 carbon atoms, wherein two hydrogen atoms of one carbon atom of the alkyl radical are replaced by a divalent C₁-C₆-alkoxyimino radical (C₁-C₆-alkyl-O—N═) as defined above.

The term “C₂-C₆-alkenyloxyimino-C₁-C₄-alkyl” refers to alkyl having 1 to 4 carbon atoms, wherein two hydrogen atoms of one carbon atom of the alkyl radical are replaced by a divalent C₂-C₆-alkenyloxyimino radical (C₂-C₆-alkenyl-O—N═).

The term “C₂-C₆-alkynyloxyimino-C₁-C₄-alkyl” refers to alkyl having 1 to 4 carbon atoms, wherein two hydrogen atoms of one carbon atom of the alkyl radical are replaced by a divalent C₂-C₆-alkynyloxyimino radical (C₂-C₆-alkynyl-O—N═).

The term “hydroxyC₁-C₄-alkyl” refers to alkyl having 1 to 4 carbon atoms, wherein one hydrogen atom of the alkyl radical is replaced by a OH group.

The term “aminoC₁-C₄-alkyl” refers to alkyl having 1 to 4 carbon atoms, wherein one hydrogen atom of the alkyl radical is replaced by a NH₂ group.

The term “C₁-C₆-alkylamino” refers to an amino group, which is substituted with one residue independently selected from the group that is defined by the term C₁-C₆-alkyl. Likewise, the term “diC₁-C₆-alkylamino” refers to an amino group, which is substituted with two residues independently selected from the group that is defined by the term C₁-C₆-alkyl.

The term “C₁-C₄-alkylamino-C₁-C₄-alkyl” refers to refers to alkyl having 1 to 4 carbon atoms (as defined above), wherein one hydrogen atom of the alkyl radical is replaced by a C₁-C₄-alkyl-NH— group which is bound through the nitrogen. Likewise, the term “diC₁-C₄-alkylamino-C₁-C₄-alkyl” refers to refers to alkyl having 1 to 4 carbon atoms (as defined above), wherein one hydrogen atom of the alkyl radical is replaced by a (C₁-C₄-alkyl)₂N— group which is bound through the nitrogen.

The term “aminocarbonyl-C₁-C₄-alkyl” refers to alkyl having 1 to 4 carbon atoms, wherein one hydrogen atom of the alkyl radical is replaced by a —(C═O)—NH₂ group.

The term “C₃-C₁₁-cycloalkyl” refers to a monocyclic, bicyclic or tricyclic saturated univalent hydrocarbon radical having 3 to 11 carbon ring members that is connected through one of the ring carbon atoms by substitution of one hydrogen atom, such as cyclopropyl (C₃H₅), cyclobutyl, cyclopentyl, cyclohexyl, cycloheptyl, cyclooctyl, bicyclo[1.1.0]butyl, bicyclo[2.1.0]pentyl, bicyclo[1.1.1]pentyl, bicyclo[3.1.0]hexyl, bicyclo[2.1.1]hexyl, norcaranyl (bicyclo[4.1.0]heptyl) and norbornyl (bicyclo[2.2.1]heptyl).

The terms “—C(═O)—C₁-C₆-alkyl”, “—C(═O)—O—C₁-C₆-alkyl” and “—C(═O)—C₃-C₁₁-cycloalkyl” refer to aliphatic radicals which are attached through the carbon atom of the —C(═O)— group.

The term “aliphatic” refers to compounds or radicals composed of carbon and hydrogen and which are non-aromatic compounds. An “alicyclic” compound or radical is an organic compound that is both aliphatic and cyclic. They contain one or more all-carbon rings which may be either saturated or unsaturated, but do not have aromatic character.

The terms “cyclic moiety” or “cyclic group” refer to a radical which is an alicyclic ring or an aromatic ring, such as, for example, phenyl or heteroaryl.

The term “and wherein any of the aliphatic or cyclic groups are unsubstituted or substituted with . . . ” refers to aliphatic groups, cyclic groups and groups, which contain an aliphatic and a cyclic moiety in one group, such as in, for example, C₃-C₈-cycloalkyl-C₁-C₄-alkyl; therefore a group which contains an aliphatic and a cyclic moiety both of these moieties may be substituted or unsubstituted independently of each other.

The term “phenyl” refers to an aromatic ring systems including six carbon atoms (commonly referred to as benzene ring.

The term “heteroaryl” refers to aromatic monocyclic or polycyclic ring systems including besides carbon atoms, 1, 2, 3 or 4 heteroatoms independently selected from the group consisting of N, O and S.

The term “saturated 3- to 7-membered carbocycle” is to be understood as meaning monocyclic saturated carbocycles having 3, 4 or 5 carbon ring members. Examples include cyclopropyl, cyclopentyl, cyclohexyl, cycloheptyl, and the like.

The term “3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms”, is to be understood as meaning both, aromatic mono- and bicyclic heteroaromatic ring systems, and also saturated and partially unsaturated heterocycles, for example:

a 3- or 4-membered saturated heterocycle which contains 1 or 2 heteroatoms from the group consisting of N, O and S as ring members such as oxirane, aziridine, thiirane, oxetane, azetidine, thiethane, [1,2]dioxetane, [1,2]dithietane, [1,2]diazetidine; and a 5- or 6-membered saturated or partially unsaturated heterocycle which contains 1, 2 or 3 heteroatoms from the group consisting of N, O and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding-ylidene radicals; and a 7-membered saturated or partially unsaturated heterocycle such as tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding-ylidene radicals.

The term “5- or 6-membered heteroaryl” or the term “5- or 6-membered aromatic heterocycle” refer to aromatic ring systems including besides carbon atoms, 1, 2, 3 or 4 heteroatoms independently selected from the group consisting of N, O and S, for example, a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

### WORKING EXAMPLES

The present invention is further illustrated by means of the following working examples.

Analytical method: HPLC Agilent 1100 Series; column: Agilent Zorbax Phenyl-Hexyl 1.8 μm 50*4.6 mm, Column Flow: 1 mL/min, time: 25 min, pressure: 20000 kPa; temperature: 20° C.; wavelength 200 nm; injector volume: 2 uL; retention time of the respective products is based on reference material.

Eluent: A: Water with 0,1 vol % H₃PO₄; B: Acetonitrile

| Time (min) | % B | Rate (mL/min) |
|---|---|---|
| 0.0 | 14 | 1.0 |
| 16.0 | 86 | 1.0 |
| 20.0 | 86 | 1.0 |
| 20.1 | 14 | 1.0 |

### Example 1) Preparation of N-(2-Fluorophenyl)-4-[5-(Trifluoromethyl)-1,2,4-Oxadiazol-3-Yl]Benzamide

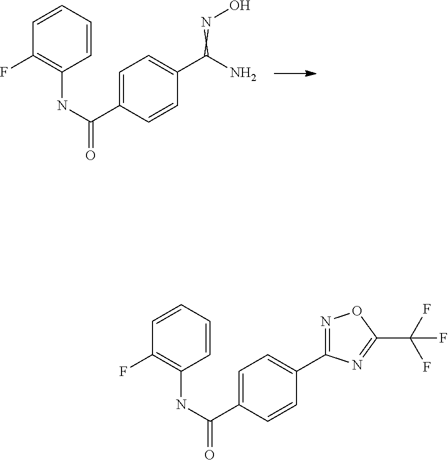

A scaleable glass vessel was charged with N-(2-fluorophenyl)-4-[(Z)-N′ hydroxycarbamimidoyl]benzamide (437.6 g (98.9% purity, 1.584 mol), methanol (1013.5 g) and ethyl trifluoroacetate (496.0 g, purity 99.8%, 3.48 mol) under an atmosphere of nitrogen. The reaction mixture was brought to 25° C. and sodium methanolate (30% w/w in methanol, 370.6 g, 2.06 mol) was added over a period of 180 minutes at a temperature of 25° C. under cooling. The resulting mixture was agitated at 25° C. for 191 minutes. Demineralized water (633.4 g) was then added at 20° C., over 15 min under agitation. The suspended solids were collected by filtration. The filter cake was washed twice with 633.4 g demineralized water. Drying under vacuum yielded the desired product as a colorless solid in 97.6% (HPLC purity: 98.1%).

### Example 2) Recycling of Methyl Trifluoroacetate by Use of Conc. Sulfuric Acid

Aqueous mother liquor obtained in Example 1) above (2019.5 g) and the first wash liquor (779.5 g) were combined and charged to a scaleable vessel fitted with a rectification column (stainless steel packaging). The content of sodium trifluoroacetate in the mixture was determined by quantitative capillary electrophoresis with 1.87 mol.

189.3 g (1.896 mol) conc. sulfuric acid (98%) have been added at room temperature. The vessel was heated to an inside temperature of 72.8° C. and the rectification was started. During the course of the rectification, the inside temperature of the vessel increased to 99.9° C. Two fractions and the content of the cooling trap were balanced. The first fraction of 1359.5 g contained 81.7% methanol, 16.47% methyl trifluoroacetate, 1.0% ethanol and 0.76% ethyl trifluoroacetate according to quantitative GC analytics. The water content was determined by Karl-Fischer Titration with 493 ppm. The cooling trap contained 4.0 g (59,4% methyl trifluoroacetate, 33.1% methanol, 1.4% ethyl trifluoroacetate, 0.3% ethanol). The yield of methyl trifluoroacetate plus ethyl trifluoroacetate in respect to the analyzed sodium trifluoroacetate content of the starting mixture was calculated with 101% (higher than 100% can be explained by uncertainties of analytics).

### Example 3) Recycling of Methyl Trifluoroacetate by Use of Conc. Hydrochloric Acid

Example 1) was repeated for the purpose of this experiment. The aqueous mother liquor (2135.8 g) and the first wash liquor (633.3 g) were combined and charged to a scaleable vessel fitted with a rectification column (stainless steel packaging). The content of sodium trifluoroacetate in the mixture has been determined by quantitative capillary electrophoresis with 1.87 mol.

214.4 g (1.88 mol) conc. hydrochloric acid (32%) were added at room temperature. The vessel was heated to an inside temperature of 74.6° C. and the rectification was started. During the course of the rectification, the inside temperature increased to 88.5° C. One fraction and the content of the cooling trap were balanced. The fraction of 1291.6 g contained 82.1% methanol, 17.2% methyl trifluoroacetate, 1.0% ethanol and 0.5% ethyl trifluoroacetate according to quantitative GC analytics. The water content was determined by Karl-Fischer Titration with 207 ppm. The cooling trap contained 3.8 g (56.8% methyl trifluoroacetate, 32.5% methanol, 3.6% ethyl trifluoroacetate, 3.0% ethanol). The yield of methyl trifluoroacetate plus ethyl trifluoroacetate in respect to the analyzed sodium trifluoroacetate has been calculated with 96.5%.

1. A process for producing an alkyl trifluoroacetate of formula I,
CF₃—C(═O)—O—R I
wherein variable R is C₁-C₆-alkyl or C₃-C₆-cycloalkyl; the process comprising reacting trifluoroacetic acid with an alkyl alcohol R—OH in an aqueous solution, wherein variable R in alkyl alcohol R—OH has the same meaning as R in the alkyl trifluoroacetate of formula I; and wherein the alkyl trifluoroacetate I is separated from the reaction medium by means of direct rectification.

2. The process according to claim 1, wherein a pH of the aqueous solution is equal to or lower than 7.

3. The process according to claim 1, wherein the rectification is carried out at a temperature of the reaction mixture between 20° C. and 100° C., at atmospheric pressure.

4. The process according to claim 1, wherein the alkyl alcohol R—OH is methanol, ethanol, n-propanol, iso propanol, n-butanol, iso-butanol, sec-butanol, tert-butanol; or mixtures thereof.

5. The process according to claim 1, wherein the alkyl alcohol R—OH is methanol.

6. A process for preparing an oxadiazole compound of formula II, 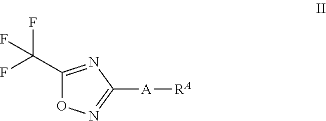
wherein
A is phenyl or a 5- or 6-membered aromatic heterocycle; wherein the ring member atoms of the aromatic heterocycle include besides carbon atoms 1, 2, 3, or 4 heteroatoms selected from N, O, and S as ring member atoms with the provision that the heterocycle cannot contain 2 contiguous atoms selected from O and S; and wherein A is further unsubstituted or further substituted with additional n identical or different radicals R^{a}; wherein
n is 0,1, 2, 3, or 4;
R^{a} is independently selected from the group consisting of halogen, cyano, C₁-C₆-alkyl, C₁-C₆-haloalkyl, C₁-C₆-alkoxy, and C₁-C₆-haloalkoxy;
R^{A} is methyl, chloromethyl, hydroxymethyl, trichloromethyl, ethyl, iso-propyl, OH, SH, cyano, halogen, CH₂F, CHF₂, 2,2,2-trifluoroethyl, cyclopropyl, —C(═O)H, —C(═NOR₂)H, —C(═O)OH, —C(═O)OR¹, —C(═W)N(R¹R²), —CR³R⁴—N(R¹R²), —CR³R⁴—OR¹, —C(═NR¹)R³, —C(═O)R³, —CR³R⁴—C(═O)OH, —CR³R⁴—C(═O)R¹, —CR³R⁴—C(═W)N(R¹R²), —O—CR³R⁴—C(═O)OH, —O—CR³R⁴—C(═O)R¹, —O—CR³R⁴—C(═W)N(R¹R²), —CR³R⁴—N(R²)—C(═W)R¹, —CR³R⁴—S(═O)₂R¹, or —CR³R⁴—N(R²)—S(═O)₂R¹; wherein
W is O or S;
R² is hydrogen, C₁-C₆-alkyl, C₂-C₆-alkenyl, C₂-C₆-alkynyl, C₁-C₆-alkoxy, C₃-C₁₁-cycloalkyl, —C(═O)—C₁-C₆-alkyl, —C(═O)—C₃-C₁₁-cycloalkyl, or —C(═O)—O—C₁-C₆-alkyl; and wherein any of the aliphatic or cyclic groups in R² are unsubstituted or substituted with 1, 2, 3, or up to the maximum possible number of identical or different radicals selected from the group consisting of halogen, hydroxy, oxo, cyano, C₁-C₆-alkyl, C₁-C₆-alkoxy, and C₃-C₁₁-cycloalkyl;
R¹ is C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, C₁-C₆-alkylamino, diC₁-C₆-alkylamino, —C(═O)—C₁-C₆-alkyl, —C(═O)—O—C₁-C₆-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl, or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms with the provision that the heterocycle cannot contain 2 contiguous atoms selected from O and S; and wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O, and S as ring member atoms with the provision that the heterocycle cannot contain 2 contiguous atoms selected from O and S; and wherein any of the above-mentioned aliphatic or cyclic groups are unsubstituted or substituted with 1, 2, 3, or up to the maximum possible number of identical or different groups R¹ª; or
R¹ and R², together with the nitrogen atom to which they are attached, form a saturated or partially unsaturated mono- or bicyclic 3- to 10-membered heterocycle, wherein the heterocycle includes beside one nitrogen atom and one or more carbon atoms no further heteroatoms or 1, 2 or 3 further heteroatoms independently selected from N, O, and S as ring member atoms with the provision that the heterocycle cannot contain 2 contiguous atoms selected from O and S; and wherein the heterocycle is unsubstituted or substituted with 1, 2, 3, 4, or up to the maximum possible number of identical or different groups R^{1a}; wherein
R^{1a} is halogen, oxo, cyano, NO₂, OH, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, —NHSO₂-C₁-C₄-alkyl, —C(═O)—C₁-C₄-alkyl, —C(═O)—O—C₁-C₄-alkyl, C₁-C₆-alkylsulfonyl, hydroxyC₁-C₄-alkyl, —C(═O)—NH₂, —C(═O)—NH(C₁-C₄-alkyl), C₁-C₄-alkylthio-C₁-C₄-alkyl, aminoC₁-C₄-alkyl, C₁-C₄-alkylamino-C₁-C₄-alkyl, diC₁-C₄-alkylamino-C₁-C₄-alkyl, aminocarbonyl-C₁-C₄-alkyl, or C₁-C₄-alkoxy-C₁-C₄-alkyl;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy; or
R³ and R⁴ together with the carbon atom to which they are bound form a cyclopropyl group;
the process comprising:
step 1: reacting an amidoxime of formula III, 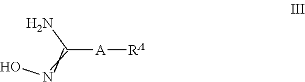
wherein the variables A and R^{A} are as defined above for compounds of formula II, with an alkyl trifluoroacetate of formula I,
CF₃—C(═O)—O—R I
wherein R is C₁-C₆-alkyl or C₃-C₆-cycloalkyl, in the presence of a base;
step 2: adding water to the reaction mixture obtained in step 1 and then separating amidoxime III from an aqueous phase;
step 3: treating combined aqueous phases obtained in step 2 with an auxiliary acid, whereas an amount of the auxiliary acid is equivalent to or more than the amount of base used in step 1;
step 4: separating the alkyl trifluoroacetate I from the aqueous phases obtained in step 3 by rectification.

7. The process according to claim 6, wherein step 1 is conducted in the presence of an auxiliary solvent.

8. The process according to claim 7, wherein the auxiliary solvent comprises methanol or ethanol.

9. The process according to claim 6, wherein the base in step 1 comprises a sodium or potassium C₁-C₆-alkoxylate.

10. The process according to claim 6, wherein the base in step 1 is sodium methoxide or sodium ethoxide.

11. The process according to claim 6, wherein the auxiliary acid in step 2 is hydrochloric acid, sulfuric acid, phosphoric acid, or nitric acid.

12. The process according to claim 6, wherein the amidoxime compound is of formula III.b, 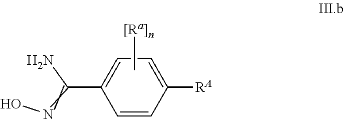
wherein n is 0 or 1, and the meaning of R^{a} and R^{A} is as defined in claim 6 for compounds of formula II, to obtain an oxadiazole compound of formula II.b, wherein the variables n, R^{a}, and R^{A} have the meaning as defined for compounds of formula III.b.

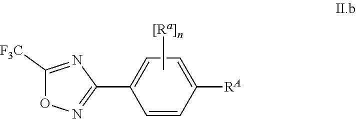

13. The process according to claim 12, wherein the variables in compounds of formulae II.b and IlI.b have the following meaning:
R^{a} is fluorine;
n is 0 or 1;
R^{A} is methyl, —C(═O)OH, —C(═O)NR¹R², —CH₂—N(R²)—C(═O)R¹, —CH₂—N(R²)—S(═O)₂R¹, 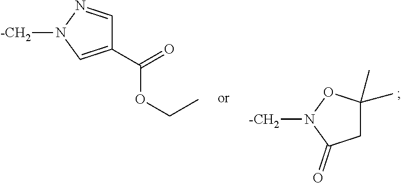
R¹ is C₁-C₆-alkly, phenyl, or cyclopropyl, wherein the phenyl ring is unsubstituted or substituted with 1, 2, 3, or 4 identical or different groups selected from halogen;
R² is hydrogen, methyl, ethyl, methoxy, ethoxy, or cyclopropyl.

14. The process according to claim 12, wherein the variables in compounds of formulae II.b and IlI.b have the following meaning:
n is 0;
R^{A} is —C(═O)N(R¹R²);
R¹ is methyl, 2-methoxyiminoethyl, bicyclo[1.1.1]pentan-1-yl, 2-fluoro-phenyl, 4-fluoro-phenyl, or 2,4-difluorophenyl; in particular methyl or 2-fluoro-phenyl;
R² is hydrogen.

15. The process according to claim 14, further comprising a step of reacting the compound of formula II.b to obtain a compound of formula IV.

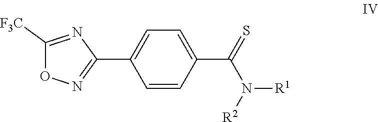

16. The process according to claim 15, wherein in compound of formula IV
R¹ is methyl or 2-fluoro-phenyl;
R² is hydrogen.
